(19) United States
(12) Patent Application Publication
(10) Pub. No.: US 2019/0102963 A1
(43) Pub. Date: Apr. 4, 2019

(54) SYSTEMS AND METHODS FOR SECURING AN OBJECT IN A VEHICLE

(71) Applicant: Toyota Motor Engineering & Manufacturing North America, Inc., Erlanger, KY (US)

(72) Inventors: Gary C. Owens, Pinckney, MI (US); June C. Owens, Pinckney, MI (US)

(73) Assignee: Toyota Motor Engineering & Manufacturing North America, Inc., Erlanger, KY (US)

(21) Appl. No.: 15/720,795

(22) Filed: Sep. 29, 2017

(51) Int. Cl. G07C 9/00; A61B 5/18; E05B 83/32; E05B 83/30

(52) U.S. Cl. CPC G07C 9/00563; A61B 5/18; G07C 2009/00769; E05B 83/30; E05B 83/32

(57) ABSTRACT

Systems and methods for securing an object in a vehicle are provided. One embodiment of a method includes receiving data related to a biometric identifier of a user, receiving data related to a physiological state of the user, and determining whether the data associated with the physiological state of the user corresponds to an undesired physiological state. In response to receiving a correct biometric identifier and determining that the physiological state of the user does not correspond to the undesired physiological state, some embodiments may be configured to grant access to a storage area of a biometric lockbox. Similarly, in response to at least one of the following: not receiving the correct biometric identifier or determining that the physiological state of the user corresponds to the undesired physiological state of the user, some embodiments may be configured to deny access to the storage area of the biometric lockbox.

### TECHNICAL FIELD

Embodiments described herein generally relate to securing an object in a vehicle and, more specifically, to a lockbox in a vehicle that utilizes a biometric identifier and a physiological state of a user to grant access to a storage area within the lockbox.

### BACKGROUND

Many drivers carry a weapon and/or a valuable item with them in a vehicle. While current solutions include lockboxes in the vehicle, these lockboxes often are secured via combination locks and/or key-access locks mounted in the center console or underneath the seat. While these current solutions may adequately secure the weapon or valuable, oftentimes these solutions are difficult to open, can be often be opened by individuals other than the owner, and do not account for whether a user should be able to access a weapon.

### SUMMARY

Systems and methods for securing an object in a vehicle are provided. One embodiment of a method includes receiving data related to a biometric identifier of a user, receiving data related to a physiological state of the user, and determining whether the data associated with the physiological state of the user corresponds to an undesired physiological state. In response to receiving a correct biometric identifier and determining that the physiological state of the user does not correspond to the undesired physiological state, some embodiments may be configured to grant access to a storage area of a biometric lockbox. Similarly, in response to at least one of the following: not receiving the correct biometric identifier or determining that the physiological state of the user corresponds to the undesired physiological state of the user, some embodiments may be configured to deny access to the storage area of the biometric lockbox.

In another embodiment, a system for securing an object in a vehicle includes a biometric lockbox that includes a storage area for receiving an object, a locking mechanism for receiving a biometric identifier of a user and a physiological sensor for detecting a physiological state of the user, and a computing device coupled to the biometric lockbox. The computing device may include logic that, when executed, causes the system to receive data related to the biometric identifier of the user, receive data related to the physiological state of the user, and in response to determining that a correct biometric identifier and a desired physiological state of the user were received, grant access to the storage area.

In yet another embodiment, a biometric lockbox includes a storage area for receiving an object, a locking mechanism that includes a biometric sensor for receiving a biometric identifier of a user and a physiological sensor for detecting a physiological state of the user, and a computing device. The computing device includes logic that, when executed, causes the biometric lockbox to receive data related to the biometric identifier of the user, receive data related to the physiological state of the user, determine whether the data associated with the physiological state of the user corresponds to an undesired physiological state, and in response to receiving a correct biometric identifier and determining that the physiological state of the user does not correspond to the undesired physiological state, grant access to the storage area.

These and additional features provided by the embodiments of the present disclosure will be more fully understood in view of the following detailed description, in conjunction with the drawings.

### BRIEF DESCRIPTION OF THE DRAWINGS

The embodiments set forth in the drawings are illustrative and exemplary in nature and not intended to limit the disclosure. The following detailed description of the illustrative embodiments can be understood when read in conjunction with the following drawings, where like structure is indicated with like reference numerals and in which:

FIG. 1 depicts a vehicle interior and computing environment for securing an object in a vehicle, according to embodiments described herein;

FIGS. 2A and 2B depict a biometric lockbox for securing an object in a vehicle, according to embodiments described herein;

FIG. 3 depicts a user interface for providing a lockbox option, according to embodiments described herein;

FIG. 4 depicts a user interface for accessing and/or configuring a biometric lockbox, according to embodiments described herein;

FIG. 5 depicts a flowchart for securing an object in a vehicle, according to embodiments described herein; and

FIG. 6 depicts a computing device for providing an object in a vehicle, according to embodiments described herein.

### DETAILED DESCRIPTION

Embodiments disclosed herein include systems and methods for securing objects in a vehicle. Some embodiments of this disclosure include a lockbox that may be installed in a center console or elsewhere in or on a vehicle. The lockbox may have a biometric access system that may be coupled to a vehicle computing device. This prevents anyone other than the driver or other authorized person that has provided the suitable biometric data (such as a fingerprint or the like) from accessing the storage area.

Additionally, by linking the lockbox to the vehicle computing device, access may be denied (to all users and/or to the driver) when the vehicle is in motion, if the vehicle detects that the person attempting to access the storage area is angry (e.g., via microphone to detect yelling; via biometric sensors to detect pulse, pupil dilation, etc.), when the vehicle is located in a predetermined area (such as at a school, airport, etc.) and/or at other times. Specifically, embodiments may be configured to determine a location of the vehicle and, in response to determining that the vehicle is located at an undesirable location, deny access to the storage area. The systems and methods for securing an object in a vehicle incorporating the same will be described in more detail, below.

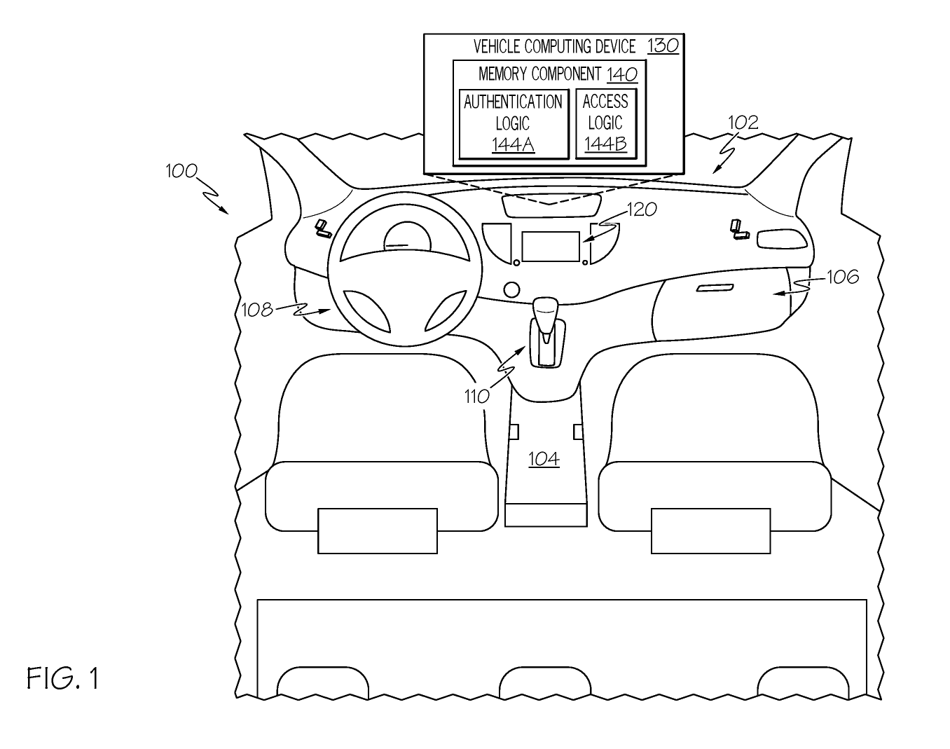

Referring now to the drawings, FIG. 1 depicts a vehicle interior 102 and computing environment for securing an object in a vehicle 100, according to embodiments described herein. As illustrated, a vehicle 100 may have a vehicle interior 102. The vehicle interior 102 may include a center console 104, a glove box 106, a steering wheel 108, and a dashboard area 110. The vehicle interior 102 may also include a vehicle head unit 120, which includes a display device, as well as a vehicle computing device 130. The vehicle computing device 130 may have a memory component 140, which stores authentication logic 144a and access logic 144b.

As described in more detail below, the vehicle head unit 120 may be communicatively coupled to a vehicle lockbox. The biometric lockbox 104 may be located in the center console 104, glove box 106, dashboard area 110, and/or at other locations in or on the vehicle 100. As such, upon execution of a processor, the authentication logic 144a may be configured cause the vehicle computing device 130 to determine whether a user attempting to access the biometric lockbox 104 is authenticated to do so. The access logic 144b may be configured to cause the vehicle computing device 130 to determine whether to provide access to the lockbox based on the physiological state of the user attempting to access storage area 208 of the lockbox (and/or location of the vehicle 100).

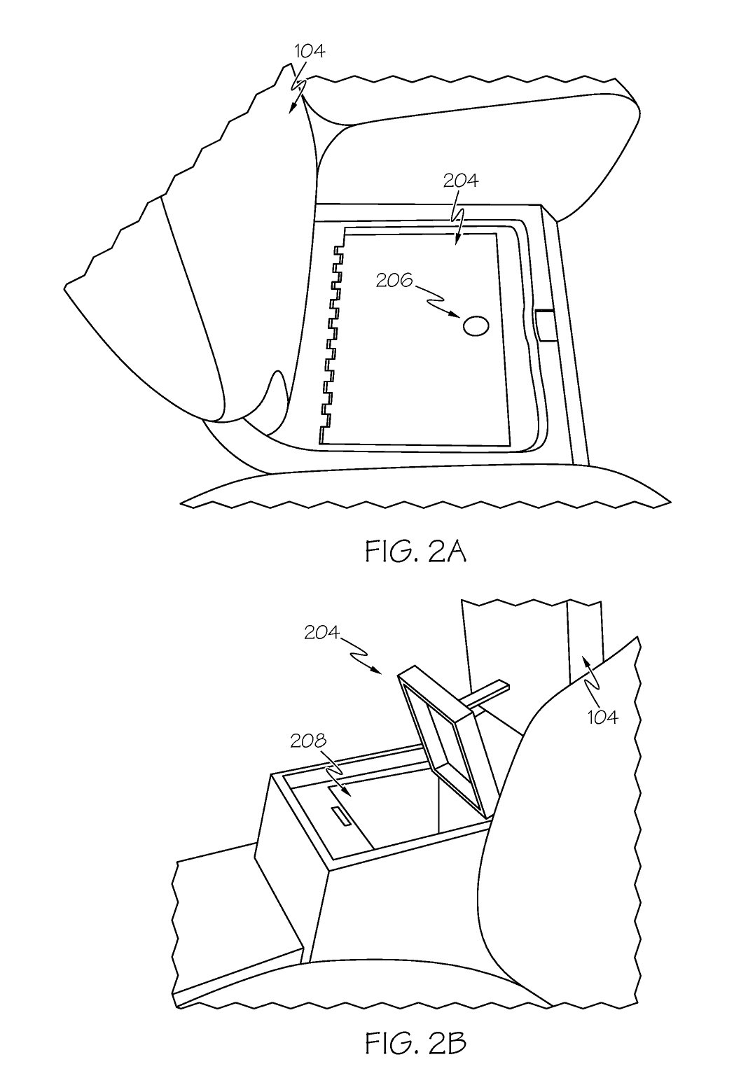

FIGS. 2A and 2B depict a biometric lockbox 204 for securing an object in a vehicle 100, according to embodiments described herein. As illustrated in FIG. 2A, the biometric lockbox 204 may be located under the center console 104, which may rotate to provide access to a surface of the biometric lockbox 204. It will be understood that, while the biometric lockbox 204 of FIG. 2A is located under the center console 104; this is merely one example. Some embodiments may be configured such that the biometric lockbox 204 is located in the glove box 106 (FIG. 1), in a compartment of the dashboard area 110 (FIG. 1), or at other areas in and/or the vehicle 100.

Regardless, the biometric lockbox 204 may include a locking mechanism 206. The locking mechanism 206 may include a latch or other lock for activating to restrict access to a storage area 208 (FIG. 2B) of the biometric lockbox 204 or deactivating to grant access to the storage area 208. The locking mechanism 206 may additionally include a biometric sensor, such as a fingerprint sensor, a retina sensor, a facial recognition sensor, a hand vein sensor, an iris sensor, a voice sensor, an ear sensor, and/or other biometric sensor. It will be understood that some of the biometric sensors may include a camera or other image capture device as well as the authentication logic 144a, which may be configured to analyze the image and authenticate a user attempting to access the storage area 208 of the biometric lockbox 204. Similarly, the biometric sensor may take the form of a microphone and/or other device for receiving audio from the user. The audio data may then be analyzed utilizing the authentication logic 144a. Regardless of whether an image capture device or a microphone is utilized, the authentication logic 144a may receive biometric data from the appropriate sensor and authenticate the user accordingly.

In addition to a biometric sensor, the locking mechanism 206 may also include a physiological sensor. The physiological sensor may be incorporated into the biometric sensor and/or may be a separate device, depending on the particular embodiment. Regardless, the physiological sensor may be coupled to the access logic 144b for determining whether a user attempting to access the storage area 208 should be granted or denied access. As an example, the physiological sensor may include at least one of the following: a retina sensor, a heartrate sensor, a voice sensor, a thermometer, and/or other physiological sensor to determine a physiological state of the user. Specifically, the physiological state of the user may be determined to assess whether the user (even if authenticated to gain access to the storage area 208) is allowed to access the storage area 208. If it is determined that the user is angry, distracted, agitated, etc. the access logic 144b may determine that access to a weapon or other object in the storage area 208 should be denied. The locking mechanism 206 may additionally include a location detection device for determining the location of the vehicle 100.

It should also be understood that some embodiments of the biometric lockbox 204 may also include a plurality of different layers of access and/or compartments. As an example, a first compartment may be configured for providing access to any authenticated user. This compartment may be utilized for storing documents, jewelry, and/or other objects that are not inherently dangerous. A second compartment may be configured for receiving weapons or other dangerous objects and thus would require authentication, as well as physiological state analysis before granting access. Accordingly, in such an embodiment, the biometric lockbox 104 may include a plurality of locking mechanisms for securing the plurality of different compartments.

It should also be understood that while the biometric sensor and the physiological sensor are depicted as being part of the biometric lockbox 204, this is also an example. These sensors may be located anywhere in the vehicle 100, such as on the steering wheel 108, on the dashboard area 110, etc., depending on the embodiment.

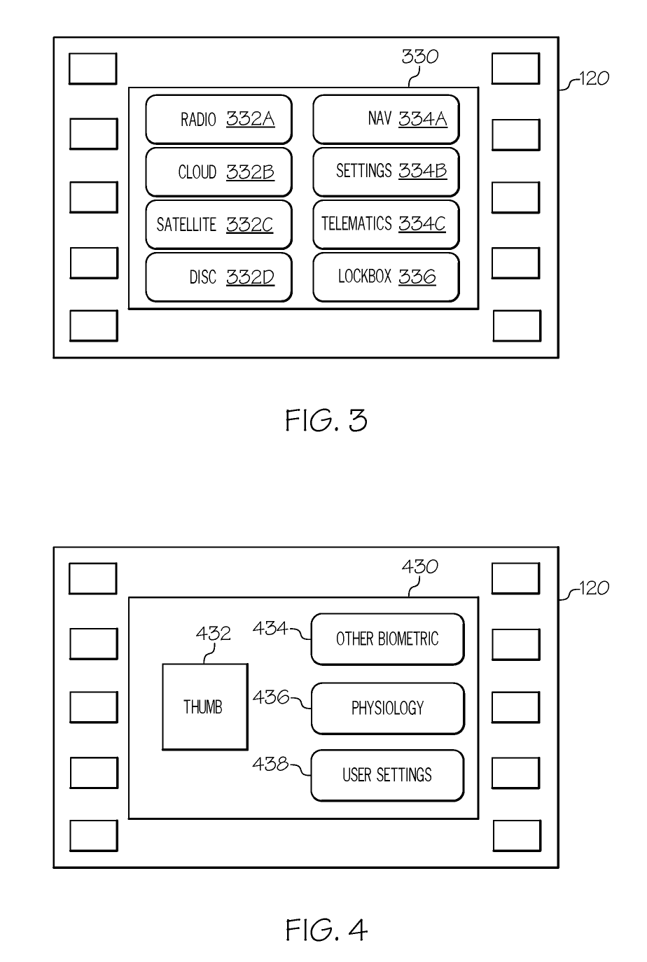

FIG. 3 depicts a user interface 330 for providing a lockbox option 336, according to embodiments described herein. As illustrated, the user interface 330 may include a radio option 332a, a cloud option 332b, a satellite option 332c, a disc option 332d, and/or other entertainment based options. The user interface 330 may also include a navigation option 334a, a settings option 334b, a telematics option 334c, and/or other information based options. A lockbox option 336 may also be provided for accessing additional options associated with the biometric lockbox 204, as depicted in FIG. 4.

FIG. 4 depicts a user interface 430 for accessing and/or configuring a biometric lockbox 204, according to embodiments described herein. As illustrated, the user interface 430 includes a thumb option 432, which includes a fingerprint sensor that is configured to receive a fingerprint of the user. As discussed above, this biometric identifier may be utilized for granting and/or denying access to the biometric lockbox 204. Additionally, the thumb option 432 may also provide a physiological sensor for detecting the emotional state of the user (such as a heart rate monitor, camera, etc.).

It will be understood that while the thumb option 432 is depicted in the user interface 430, the biometric sensor and/or the physiological sensor may be located anywhere in the vehicle 100, depending on the embodiment. As an example, some embodiments may be configured to receive a voice command, such as “open the lockbox.” In response a voice analysis may be performed to identify the user (biometric identifier), as well as determine whether the physiological state is acceptable for accessing the storage area 208. Similarly, some embodiments may utilize a plurality of different biometric sensors and/or physiological sensors to grant access. Referring to the example above, if the physiological state cannot be verified, the system may then take the user's pulse such as via a pulse sensor on the steering wheel 108. Some embodiments may be configured to capture a plurality of biometric identifiers and/or physiological state identifiers and utilizing at least a portion of those to grant or deny access to the storage area 208.

Also included in the user interface 430 are an other biometric option 434, a physiology option 436, and a user settings option 438. In response to selecting the other biometric option 434, the user may select and/or configure other biometric sensors. As an example, if the vehicle 100 includes a camera, in response to selection of the other biometric option 434, the user may download retinal scanning software for configuring retinal scanning authentication. Options may also be provided for setting up user biometric identifiers for current user and/or setting up biometric identifiers for new users.

In response to selection of the physiology option 436, the user may configure the physiological requirements for accessing the storage area 208 of the biometric lockbox 204. As an example, the user may determine the acceptable states for accessing the storage area 208, as well as sample the physiological state of the user at various times to classify the desired physiological state. In response to selection of the user settings option 438, other user settings may be configured.

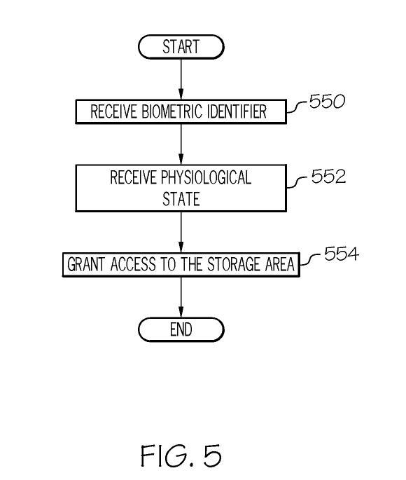

FIG. 5 depicts a flowchart for securing an object in a vehicle 100, according to embodiments described herein. As illustrated in block 550, data related to the biometric identifier of the user may be received, were the biometric identifier was received from the user to access a storage area 208 of a vehicle mounted biometric lockbox 204. In block 552, data associated with the physiological state of the user may be received. In block 554, in response to receiving a correct biometric identifier and a desired physiological state of the user, access may be granted to the storage area 208 of the internal compartment.

It should be understood that while determining that the user is in the desired physiological state (coupled with a correct biometric identifier) may allow access to the storage area 208, receiving an undesired physiological state or not receiving the desired physiological state may cause embodiments to deny access to the storage area 208. Similarly, receiving an incorrect biometric identifier or not receiving the correct biometric identifier may cause embodiments to deny access to the storage area 208.

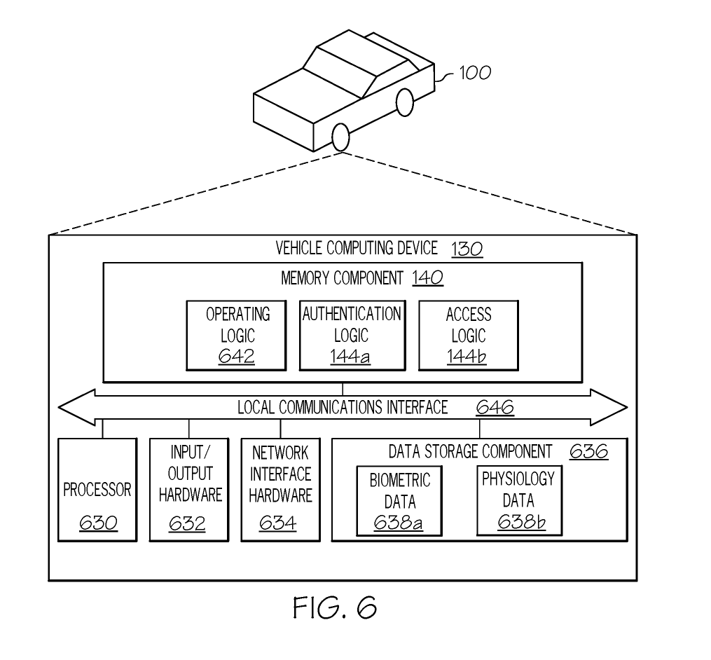

FIG. 6 depicts a vehicle computing device 130 for providing an object in a vehicle 100, according to embodiments described herein. The vehicle computing device 130 includes a processor 630, input/output hardware 632, the network interface hardware 634, a data storage component 636 (which stores biometric data 638a, physiology data 638b, and/or other data), and the memory component 140. The memory component 140 may be configured as volatile and/or nonvolatile memory and as such, may include random access memory (including SRAM, DRAM, and/or other types of RAM), flash memory, secure digital (SD) memory, registers, compact discs (CD), digital versatile discs (DVD), and/or other types of non-transitory computer-readable mediums. Depending on the particular embodiment, these non-transitory computer-readable mediums may reside within the vehicle computing device 130 and/or external to the vehicle computing device 130.

The memory component 140 may store operating logic 642, the authentication logic 144a and the access logic 144b. The authentication logic 144a and the access logic 144b may each include a plurality of different pieces of logic, each of which may be embodied as a computer program, firmware, and/or hardware, as an example. A local communications interface 646 is also included in FIG. 6 and may be implemented as a bus or other communication interface to facilitate communication among the components of the vehicle computing device 130.

The processor 630 may include any processing component operable to receive and execute instructions (such as from a data storage component 636 and/or the memory component 140). As described above, the input/output hardware 632 may include and/or be configured to interface with the components of the vehicle 100, such as the vehicle head unit 120, the sensors, the motors, etc.

The network interface hardware 634 may include and/or be configured for communicating with any wired or wireless networking hardware, including an antenna, a modem, LAN port, wireless fidelity (Wi-Fi) card, WiMax card, Bluetooth chip, USB card, mobile communications hardware, and/or other hardware for communicating with other networks and/or devices. From this connection, communication may be facilitated between the vehicle computing device 130 and other computing devices, such as via the internet, to provide the functionality described herein.

The operating logic 642 may include an operating system and/or other software for managing components of the vehicle computing device 130. As also discussed above, the authentication logic 144a may reside in the memory component 140 and may be configured to cause the processor 630 determine whether received biometric data meets predetermined requirements for authenticating a user for access to the biometric lockbox 204. Similarly, the access logic 144b may be utilized to receive sensor data; determine a physiological state of the user; and determine whether that physiological state meets predetermined requirements for granting access to the biometric lockbox 204.

It should be understood that while the components in FIG. 6 are illustrated as residing within the vehicle computing device 130, this is merely an example. In some embodiments, one or more of the components may reside external to the vehicle computing device 130. It should also be understood that, while the vehicle computing device 130 is illustrated as a single device, this is also merely an example. In some embodiments, the authentication logic 144a and the access logic 144b may reside on different computing devices. As an example, one or more of the functionality and/or components described herein may be provided by the vehicle computing device 130 and/or other device, which may be coupled to the vehicle computing device 130 via a network, such as the internet.

Additionally, while the vehicle computing device 130 is illustrated with the authentication logic 144a and the access logic 144b as separate logical components, this is also an example. In some embodiments, a single piece of logic may cause the vehicle computing device 130 to provide the described functionality.

As illustrated above, various embodiments for securing an object in a vehicle are disclosed. These embodiments provide increased security by only allowing one or more predetermined users to authenticate for access to the storage compartment of the lockbox. These embodiments also determine a physiological state of the user. By determining if the physiological state meets a predetermined stress level or other predetermined state, these embodiments can ensure that the user is in a calm state before granting access to the storage compartment.

While particular embodiments and aspects of the present disclosure have been illustrated and described herein, various other changes and modifications can be made without departing from the spirit and scope of the disclosure. Moreover, although various aspects have been described herein, such aspects need not be utilized in combination. Accordingly, it is therefore intended that the appended claims cover all such changes and modifications that are within the scope of the embodiments shown and described herein.

It should now be understood that embodiments disclosed herein include systems, methods, and non-transitory computer-readable mediums for securing an object in a vehicle. It should also be understood that these embodiments are merely exemplary and are not intended to limit the scope of this disclosure.

1. A method for securing an object in a vehicle comprising:
sampling a physiological state of a user at various times to classify a predetermined undesired physiological state;
receiving data related to a biometric identifier of the user;
receiving data related to a current physiological state of the user;
determining whether the biometric identifier of the user corresponds with an authorized user;
determining whether the data associated with the current physiological state of the authenticated user corresponds to the predetermined undesired physiological state;
in response to determining that the received data related to the biometric identifier corresponds with the authorized user and determining that the current physiological state of the user does not correspond to the predetermined undesired physiological state, granting access to a storage area of a biometric lockbox; and
in response to at least one of the following: determining that the received data related to the biometric identifier does not correspond with an authorized user or determining that the current physiological state of the user corresponds to the predetermined undesired physiological state of the user, denying access to the storage area of the biometric lockbox.

2. The method of claim 1, wherein the physiological state is determined from at least one of the following: a retina sensor, a heartrate sensor, a voice sensor, or a thermometer.

3. The method of claim 1, wherein the biometric identifier is received via at least one of the following: a fingerprint sensor, a retina sensor, a facial recognition sensor, a hand vein sensor, an iris sensor, a voice sensor, or an ear sensor.

4. The method of claim 1, wherein the biometric lockbox is located in at least one of the following: a center console of the vehicle, a glove box of the vehicle, or a dashboard area of the vehicle.

5. The method of claim 1, further comprising granting access to a compartment of the storage area upon receiving the correct biometric identifier.

6. The method of claim 1, further comprising determining a location of the vehicle and, in response to determining that the vehicle is located at an undesirable location, denying access to the storage area.

7. (canceled)

8. A system for securing an object in a vehicle comprising:
a biometric lockbox that includes a storage area for receiving an object, and a locking mechanism that includes a lock, a biometric sensor for detecting a biometric identifier to authenticate a user and a physiological sensor for detecting a physiological state of an authenticated user to determine whether the authenticated user is in a mental and physical condition to access the storage area; and
a computing device coupled to the biometric lockbox that includes logic that, when executed, causes the system to perform at least the following:
sample the physiological state of the user at various times to classify a predetermined desired physiological state;
receive data related to the biometric identifier of the user;
receive data related to the physiological state of the user; and
in response to determining that a correct biometric identifier and the predetermined desired physiological state of the user were received, deactivate the lock to grant access to the storage area.

9. The system of claim 8, wherein the physiological sensor includes at least one of the following: a retina sensor, a heartrate sensor, or a thermometer.

10. The system of claim 8, wherein the biometric sensor includes at least one of the following: a fingerprint sensor, a retina sensor, a facial recognition sensor, a hand vein sensor, an iris sensor, a voice sensor, or an ear sensor.

11. The system of claim 8, wherein the biometric lockbox is located in at least one of the following: a center console of the vehicle, a glove box of the vehicle, or a dashboard area of the vehicle.

12. (canceled)

13. The system of claim 8, wherein the storage area includes a compartment to which access is granted upon receiving the correct biometric identifier.

14. The system of claim 8, wherein in response to a determination that at least one of the following: the physiological state of the user does not correspond to the predetermined desired physiological state or not receiving the correct biometric identifier, the logic causes the system to deny access to the storage area.

15. A biometric lockbox for securing an object in a vehicle comprising:
a storage area for receiving an object;
a locking mechanism that includes a biometric sensor for receiving a biometric identifier of a user for authenticating the user and a physiological sensor for detecting a physiological state of an authenticated user to determine whether the authenticated user is in a physical and mental state to access the object; and
a computing device that includes logic that, when executed, causes the biometric lockbox to perform at least the following:
sample the physiological state of the user at various times to classify a predetermined undesired physiological state;
receive data related to the biometric identifier of the user;
receive data related to the physiological state of the user;
determine whether the data associated with the physiological state of the user corresponds to the predetermined undesired physiological state; and
in response to receiving a correct biometric identifier and determining that the physiological state of the user does not correspond to the predetermined undesired physiological state, grant access to the storage area.

16. The biometric lockbox of claim 15, wherein in response to a determination that at least one of the following: the physiological state of the user corresponds to the predetermined undesired physiological state or not receiving the correct biometric identifier, the logic causes the biometric lockbox to deny access to the storage area.

17. The biometric lockbox of claim 16, wherein the physiological sensor includes at least one of the following: a retina sensor, a heartrate sensor, or a thermometer.

18. The biometric lockbox of claim 15, wherein the biometric sensor includes at least one of the following: a fingerprint sensor, a retina sensor, a facial recognition sensor, a hand vein sensor, an iris sensor, a voice sensor, or an ear sensor.

19. (canceled)

20. The biometric lockbox of claim 15, wherein the biometric lockbox is located in one of the following: a center console of the vehicle and a glove box of the vehicle.

21. The biometric lockbox of claim 15, wherein the storage area includes a compartment to which access is granted upon receiving the correct biometric identifier.
